(19) United States
(12) Patent Application Publication
(10) Pub. No.: US 2006/0287629 A1
(43) Pub. Date: Dec. 21, 2006

(54) Jet injector with a bi-stable spring

(75) Inventor: Anthony William Denne, Cambridge (GB)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 10/532,521

(22) PCT Filed: Oct. 31, 2003
(86) PCT No.: PCT/GB03/04706

§ 371 Date: Apr. 25, 2005

(30) Foreign Application Priority Data
Nov. 1, 2002 (GB) 0225498.5
Nov. 26, 2002 (GB) 0227518.8

(51) Int. Cl. A61M 5/30

(52) U.S. Cl. 604/68

(57) ABSTRACT

A jet injector is described in which a rigid tube (1) terminates in a nozzle (15) at one end and a non return valve (46) at the other. There is a hole (3) in the tube wall and an elastomeric liner (11) within the rigid tube (1). There is also a C spring attached to a rotatable mass (24), which in turn bears a piston (26). The spring is bi-stable. It may be cocked manually and triggered by pressure on the skin such that the mass is accelerated and the piston impacts on the elastomeric liner to produce a high pressure transient. This in turn produces a liquid jet that pierces the skin and permits administration of the drug through the hole so formed, using a the piston in a standard syringe.

PCT/GB02/02633 describes a jet injector in which there is a rigid tube terminating at one end in a nozzle and at the other in a constriction which leads to the main drug supply. A portion of the rigid tube is formed as a flexible window. There is an over centre spring and an end thrust beam which may compress the window to cause a high speed flow through the nozzle. The device suffers a number of problems. Priming the pump is unstable, the spring acting in tension stores insufficient energy, the flexible window tears from its mount and causes inefficient energy transfer, the spring and beams carry insufficient momentum, the nozzle form tends to close the entrance to the track through the skin and energising by pressing against the skin of the patient is somewhat uncomfortable. The present specification details radical improvements to the device

According to the present invention, there is an injector comprising a rigid tube with an outlet at one end and a non-return valve at the other end, a hole in the tube wall, an elastomeric liner within the rigid tube and a piston arranged to impact the elastomeric liner through the hole in the tube to produce a high pressure transient. The injector includes a spring member arranged to act upon the piston to cause the piston to impact the liner. The piston is attached to a mass which is capable of being accelerated by the spring member. The spring member is bi-stable. It may be manually energised to a latched position and may then be triggered by pressure against the skin of a patient. The spring member comprises an arcuate lamina which is deformable by bending to decrease in curvature. One end of the spring member is pivotable in a transverse channel of the rigid tube. The other end of the spring member is connected to the mass. The mass may be triangularly shaped. An apex of the triangularly shaped mass is pivotable in a retaining groove of the rigid tube. The mass bears the piston which fits within the hole in the rigid tube for impacting the elastomeric liner. The elastomeric liner is oversize or axially compressed to provide a seal within the rigid tube. The elastomeric liner may be axially compressed between a shoulder within the rigid tube and an oversize plug which comprises a channel and is retained by friction therewithin. The plug may be a cylinder comprising one or more flats or a helical groove, such that the fit of the plug within the bore of the tube defines one or more capillary feed channels. The walls of the compressed elastomeric liner may cover the capillary channels formed by the flats or helical groove, to form a non-return valve biased in the closed position. Alternatively, the plug may be an oversize capillary tube which retains or compresses the elastomeric liner and is itself retained by frictional forces. The injector may include a blind elastomeric tube with a transverse slot off-axis, which forms a non-return valve with the retaining capillary tube. A conical cross-section of the blind termination of the elastomeric tube may bias the valve into a closed position. The capillary tube may be sharpened to pierce the rubber septum of an ampoule, to provide a supply of liquid drug to the tube. The elastomeric liner is preferably made of silicone rubber. The silicone rubber may be filled with a proportion of silicone oil to provide intrinsic lubrication. The injector outlet may be a nozzle and the surface surrounding the outlet may be saddle-shaped. The cross-section of the saddle-shaped outlet is preferably rectangular. The cross-section of the saddle-shaped outlet may increase in size with distance from the axis thereof. The injector may include a retractor spring which partially removes the piston from the hole in the rigid tube wall, when the injector is in its quiescent state, so that the elastomeric liner does not take a compression set. The retractor spring is preferably low rate and does not retract the piston beyond a given point, such that the elastomeric liner is not extruded through the hole in the rigid tube wall by pressure from an associated injection syringe, in use.

The present injector comprises a rigid plastic tube with a nozzle at one end and a transverse hole through the tube wall. The tube contains a tubular liner of silicone rubber which is retained between a shoulder within the tube and an interference fit plug inserted into the end of the tube. The liner and hole are in register and compression of the liner between shoulder and plug ensures a water tight seal. The plug and liner are so fashioned that they form both a non return valve and a capillary drug feed tube. There is a C spring, one end of which may rotate in a groove in the rigid tube wall. The other is rigidly attached to an elongated mass such that rotation of the mass extends the spring. There is a piston attached to the mass such that contraction of the spring causes the piston to enter the hole in the rigid plastic tube and impact on the silicone rubber liner. There is a detent such that the spring is bi-stable. It may be extended manually to energise it. It may then be triggered by gentle pressure on the skin There is a retractor spring that retains the piston partially out of the tube in the quiescent state to prevent compression set in the silicone liner and to provide support to the liner under pressure from the associated syringe during drug delivery.

Triggering the spring causes the spring energy to accelerate an attached mass. At peak velocity, a piston attached to the mass penetrates the hole in the rigid tube wall and impacts with the silicone liner to produce a high pressure transient. This transient travels down the liquid drug filled rigid tube to the nozzle where it generates a high velocity jet. This jet cuts a track through the skin. The outer surface surrounding the nozzle keeps the entrance to the track open and in register with the nozzle so that the main dose may be fed through this hole in the skin using the piston in an attached syringe.

The silicone liner is a key element of the device. A silicone window within the wall of the rigid tube will tend to move inwards under the pressure of a piston. If the window is bonded, the bond will tend to tear under the high stresses entailed in forming a high pressure seal between the piston and hole. If the window is mechanically held, movement will be inevitable. The pressure generated at impact is extremely high so the frictional force between the silicone window and hole in the rigid tube is very high and work done by the piston against this friction is very significant. Indeed, it may absorb 90% of the system energy. By using a liner within the tube, there is no possibility of the piston doing work against frictional loading between the silicone and the tube wall. The strain in the silicone rubber at the seal between the piston and the rigid wall may be quite high. There are grades of silicone that extend 1000% before failure and this may be extended by inclusion of silicone oil in the rubber. An oil filled rubber will release oil under high strain and this free oil will form a lubrication film. Indeed, with a film of oil between the rubber and rigid walls, most of the silicone rubber will be under just isostatic pressure.

The non return valve within the injector serves three important functions. It is necessary to expel air from the injector system by flushing in the normal fashion. This is to prime the injector pump as well as preventing the formation of an air embolism within the patient. The pump volume is extremely small so the slightest spring back of the piston after flushing will cause the injector to fill with air again.

The non return valve inhibits this. Biasing the valve shut will clearly enhance efficiency. Secondly, by ensuring that flow from the pump is unidirectional during jet generation, rather than bidirectional as in PCT/GB02/02633, more fluid is available for the jet and jet duration may last longer as there is no shunt from flow back into the syringe reservoir. The third function is more subtle. If there is bidirectional flow out of the pump working volume, at the end of the jet generation phase there will be a very large negative pressure within the pump cavity. This will result in cavitation. Diffusion of dissolved air into this cavity will form a stable bubble that may compromise priming of the pump for a subsequent shot. With a non return valve, the low pressure will simply open the valve and induce flow from the syringe reservoir. This not only inhibits cavitation but will marginally prolong the piercing jet flow.

The external profile of the nozzle is shaped to perform three important functions. There is a peripheral edge feature that generates a high contact pressure with the skin. This acts as an effective hydraulic seal that prevents leakage of drug during delivery of the main dose. By arranging that the area of this seal is very small and the skin forms around a small radius edge, it may be ensured that the hydraulic seal pressure will always greatly exceed the drug delivery pressure from the syringe. In this manner, a wet shot is impossible.

The second function of the high pressure seal is to maintain alignment of the hole with the nozzle during drug delivery. The local high pressure of the seal at the edge implies a pressure gradient that will pump away any extraneous liquid on the skin that could act as a lubricant. The contact at the seal will be with dry skin therefore and there will be a high coefficient of friction. This will effectively maintain registration of the skin with the nozzle. Because the seal has comparable size to the nozzle effective alignment of nozzle and track will be maintained.

The third function is to keep the orifice of the track open under pressure. Mechanically, the skin tends to act as a thick membrane. Depressing it locally with the injector will cause the outer surface to go into compression, so effectively sealing the entry to the track. By creating a saddle shaped outer surface to the nozzle, the outer surface of the skin may be locally placed in tension so that the orifice to the track is held open. A rectangular cross section is more effective than a gently curved anticlastic form. This is yet more effective if the width of the channel increases with distance from the axis of the nozzle. The skin bulges into the rectangular channel which places the surface in tension. The skin may touch the roof of the channel to form a hydraulic seal and any trapped air is pumped into the quasi triangular spaces formed between the skin and the right angled corners of the channel. A 2:1 rectangle may stretch the skin by 100% compared with 50% for a semicircular channel.

The spring performance is vital and quite subtle. The spring in PCT/GB02/02633 stores energy in tension. While this is very efficient on volume, it implies very thin spring members as energy storage capacity diminishes rapidly with additional bending strain. Because of speed constraints, the size of the spring is limited. The total spring energy available was significantly limited. Within a given size constraint, a C spring may store a significantly larger amount of energy in pure bending strain. Further, by tapering the spring in either thickness or width, the maximum permissible surface strain can be maintained throughout most of the mass of the spring. The tip of the C spring may point approximately in the direction of impact so the spring will be very rigid to the compression load at impact and the momentum of the spring will contribute to the impact loading.

While a C profile offers advantages, the spring profile may be J or L shaped or indeed any arbitrary arcuate form It is convenient to use two springs symmetrically disposed about the rigid tube and mechanically joined at either end. It is found that it is desirable to fabricate the spring as an injection moulded component and polyether ether ketone [PEEK] is found to be the optimal polymer for this purpose.

The spring energy transforms to kinetic energy, but the load generated at impact equals the rate of destruction of momentum It is vital therefore that the mass of the system is optimised to provide efficient energy transfer. By connecting the spring to a mass which in turn connects to a piston, the energy match can be optimised. In PCT/GB02/02633, the mass of the end thrust beams is constrained by speed and pressure requirements, so there is very little scope for effective matching.

The mass attached to the C spring may be essentially triangular in form. If one apex attaches to the spring, another may rotate in transverse groove in the outer wall of the rigid tube, so that rotation of the mass extends and energises the spring. Continued rotation will eventually reduce the spring length, so a stop just past maximum extension provides a latch point. If the spring rotates toward the nozzle during extension, pressure from the skin of the patient may return the spring through the maximum extension point and initiate spontaneous collapse. In this manner, pressure against the skin of the patient can trigger the formation of a piercing liquid jet. The relative geometry of the nozzle and spring may determine that the nozzle is securely located and sealed to the skin before triggering. Similarly the trigger load may be determined by the choice geometry.

The load mass rotates to energise the spring. The unattached end of the spring rotates in a groove in the outer wall of the rigid tube and the spring ends rotate through a given angle on extension of the spring. With careful design, all these rotations may be matched so that there is no significant moment applied to the spring in the latched configuration.

Most silicone rubbers will creep under constant load especially if oil filled. If the piston remains fully inserted in the quiescent state, such creep would provide a much reduced displacement on the pumping stroke. An extension to the main spring may bear on the rigid tube in such a way that in the quiescent state, the piston is partly retracted from the rubber liner to prevent such creep occurring. If the piston is maintained within the hole in the rigid tube, it may act as a support for the silicone liner when the main dose is fed by hydraulic pressure generated by the piston in the syringe. This retractor spring may be arranged to be low spring rate compared with the main spring, so that it does not significantly affect the impact mechanics.

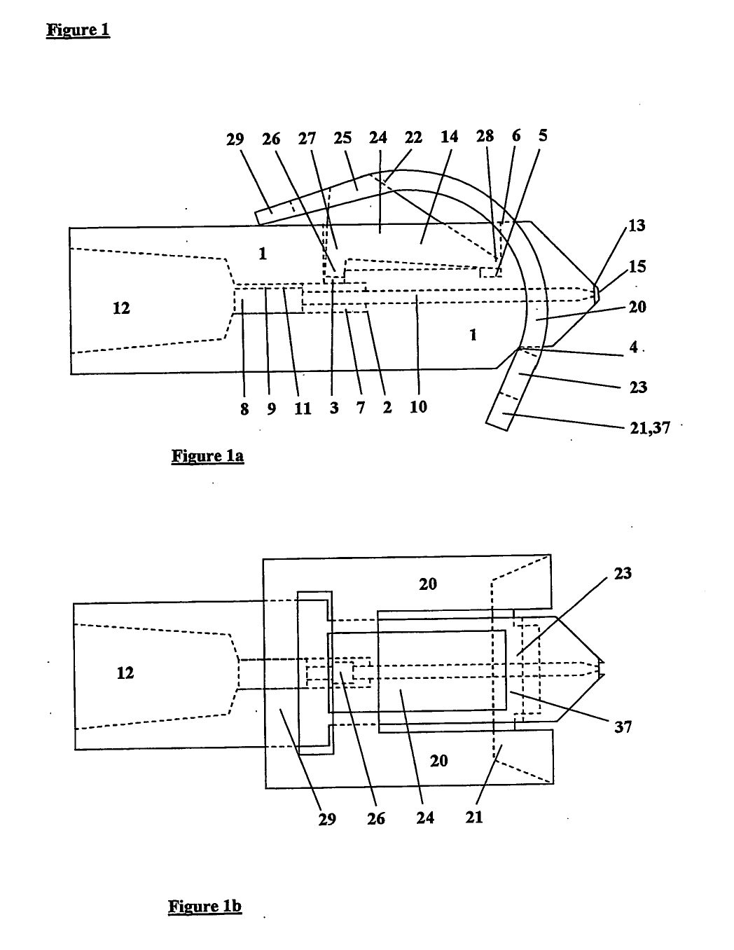

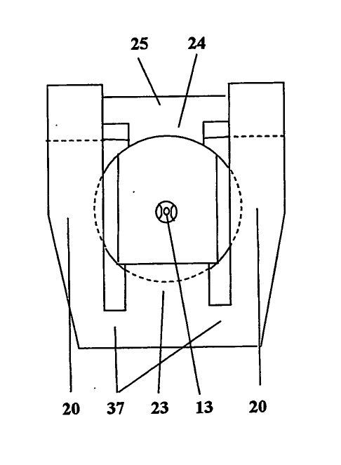

The preferred embodiment is shown schematically in FIGS. 1 to 5.

FIG. 1abc show three orthogonal views of the injector assembly in the quiescent state.

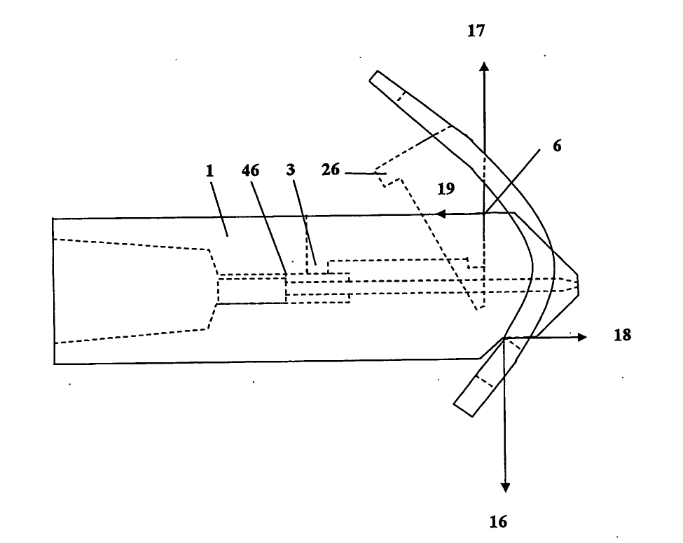

FIG. 2 shows the spring latched in the energised state

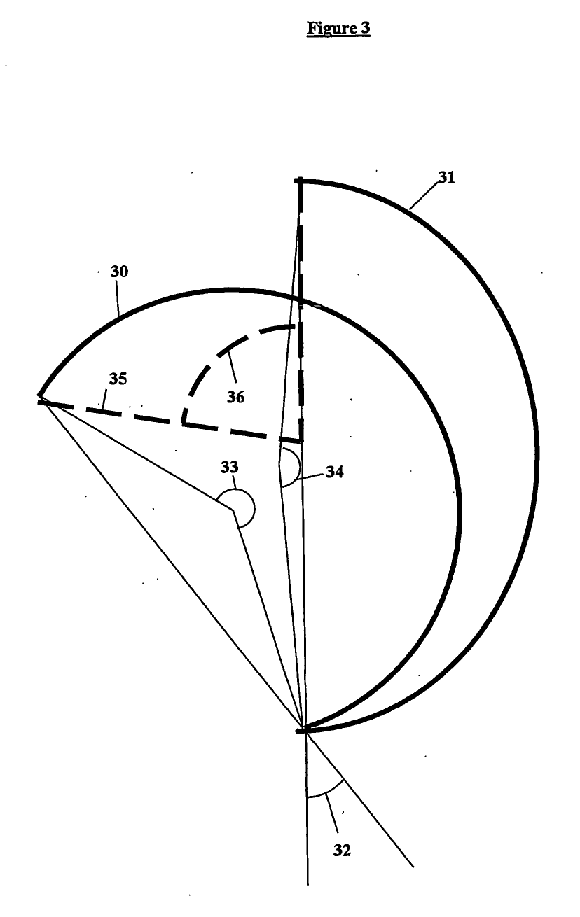

FIG. 3 illustrates the rotational mechanics of the spring

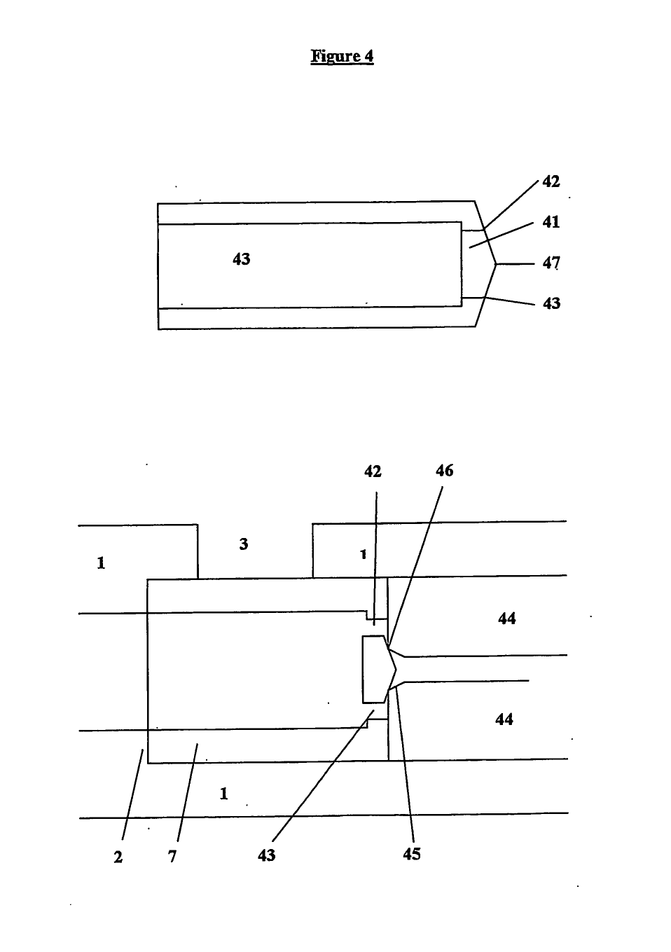

FIG. 4 shows a valve structure suitable for use with an ampoule

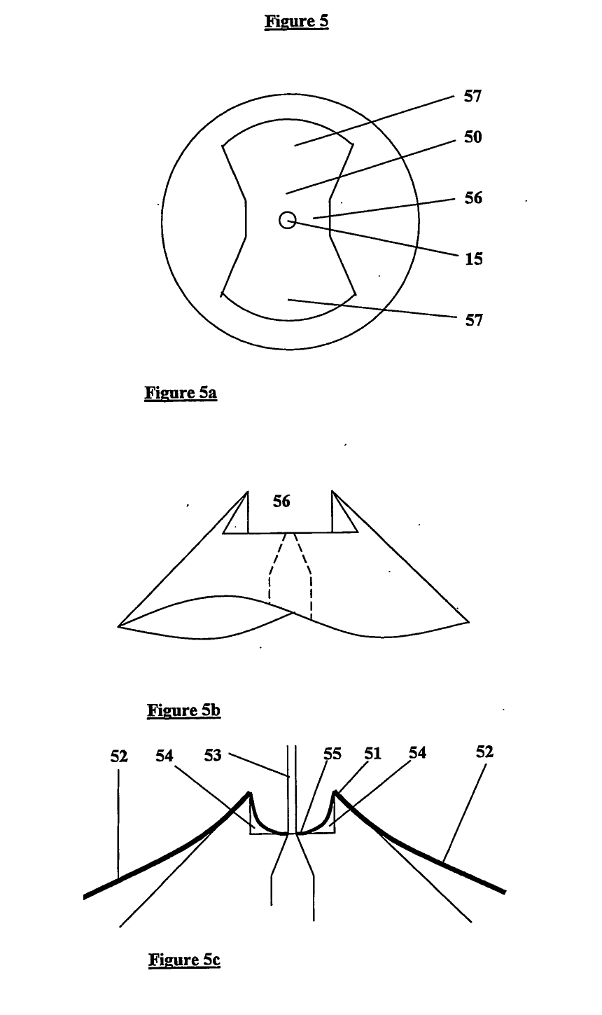

FIG. 5 shows the saddle surface around the nozzle.

FIG. 1 shows a rigid tube, 1, injection moulded in polysulphone. It comprises a nozzle, 15, a shoulder, 2, in the bore, 10, a transverse hole, 3, through the rigid tube wall, spring retaining grooves, 4 and 5 in the outer wall of the body, a latch stop, 6, a saddle shaped surface, 13, around the nozzle, 15, a spring retaining channel, 14, and a Luer taper fitting, 12. There is a tubular silicone liner, 7, within the bore of the rigid tube and an interference fit retaining plug, 8, which comprises one or more flats, 9, that form in conjunction with the bore of the rigid tube, 10, capillary feed tubes, 11. There are two springs, 20, joined at either end, 21, 22 to form a single unit with a plane of symmetry through the tube axis. At the free end, 21, the spring bridge, 37, connects to a tab, 23, which in turn bears on the retaining groove, 4. This tab permits combining a bearing forward of the latch stop, 6, with a longer spring that extends well behind the latch stop, 6. At the other end, 22, the springs join through a rigid connection to one apex, 25, of the triangular mass, 24. A small piston, 26, is attached near a second apex, 27, and the third apex, 28, bears in the retaining groove, 5. At the tip of the spring structure, there is formed a retractor spring, 29, that bears on the outer wall of the rigid tube.

The injector is assembled by inserting the slightly undersized silicone liner tube, 7, into the bore, 10, of the rigid tube, 1, then inserting the oversize retaining plug, 8. The retaining plug compresses the silicone to form a hydraulic seal against the shoulder, 2, and the bore, 10, of the rigid tube, 1. The thickness of the silicone tube, 7, is such that it seals the capillary tubes, 11, to form an effective non return valve, 46. The axial compression in the silicone tube, 7, biases the valve shut. The geometry of this valve is such that it may open at 0.1 bar pressure from the dispensing syringe, yet withstand in excess of 100 MPa reverse pressure during the jet forming pressure transient. The silicone rubber is preferably a highly extensible medical grade filled with 20% silicone oil. As explained above, the oil may act as a lubricant during the high strains of jet ejection. It will also inhibit possible bonding of the silicone to the plug or bore of the rigid tube.

The spring may be extended over a specially shaped former to ensure uniform strain then relaxed into position on the rigid tube. The lip on the groove, 5, ensures that the grooves, 4 and 5, together with latch stop, 6, retain the spring thereafter.

FIG. 2 shows the spring in the energised state. The reactions, 16, 17, from the grooves, 4 an 5, produce a couple that is balanced by a compensating couple from the groove, 18, and the latch stop, 19. This provides a stable trigger position.

FIG. 3 illustrates the relative rotations in the spring. The C spring is represented by a heavy line, 30, in its initial state and, 31, in the energised state. The spring rotates through angle, 32, during energising. On extension, the angle subtended by the ends of the spring reduces from 33 to 34. The line 35 represents the upper surface of the mass, 24. This rotates by 36 during energising. Clearly for the spring to be free of an applied moment when fully energised 36=32+33−34. There are clearly sufficient unconstrained variables for this condition to be met.

FIG. 4 shows the valve design for an ampoule fitting. The free silicone liner, 40, is shown in axial section in FIG. 4a. It is a blind tube in which the blind end, 41, has a conical cross section, 47. The blind end has two parallel off-axis transverse cuts, 42 and 43. FIG. 4b shows the silicone liner within the rigid tube bore, 10, axially compressed between an interference fit stainless steel capillary tube, 44, and the shoulder, 2. There is a conical counterbore to the stainless steel capillary tube, 45. The axial compression of the liner causes radial expansion of the blind end which opens the two transverse cuts, 42, 43, to form valve outlet ports. The conical rubber moulding, 47, is forced against the counterbore, 45, to provide a non-return valve seal, 46, which is biased closed.

FIG. 5 shows the saddle profile, 50, around the nozzle, 15. FIG. 5a shows the arrangement in plan form and FIG. 5b in elevation. FIG. 5c shows a section through the plane of symmetry with the injector in use. The tips of the walls, 51, both seal and locate the skin, 52. The skin bulges into the rectangular channel, 56, under pressure and the surface goes into tension. At the centre of the channel, the skin seals to the nozzle and any trapped air is pumped in the voids, 54, at either side. The jet cuts a hole through the skin, 53. To relieve tensile strain, the entrance to the hole, 55, stays open. It is found that operation is improved if the saddle channel broadens with distance from the nozzle, 57.

In operation, a syringe bearing a Luer taper fitting is filled with liquid drug and is fitted to the Luer fitting on the jet injector. The assembly is held vertical and syringe is tapped in the usual manner to bring any air bubbles to the top of the system. The air is flushed from the syringe and the injector by flushing with liquid drug by displacing the piston in the syringe. The spring on the injector is then cocked manually. The assembly is held by the syringe piston and in pressed against the skin of the patient at the injection site. The nozzle seals against the skin of the patient and localises the nozzle in relation to the skin. The spring is then triggered by the pressure against the skin of the patient. This accelerates the triangular mass. The piston attached to the mass penetrates the hole in the rigid tube and impacts on the silicone liner. This creates a high hydraulic pressure of approximately 100 MPa which propagates as a shock front down the rigid tube. At the nozzle, this pressure wave generates a high speed jet of liquid drug which is travelling at approximately Mach 2 in human flesh. The velocity rise time at the leading edge of this jet is approximately 1-2 microseconds. The jet pierces the skin of the patient. The boundary layer of liquid drug is of course stationary in contact with the skin. The passage of the finite jet is therefore limited by the losses to the boundary layer. The high speed leading edge with therefore penetrate the toughest of epidermis, but the depth of penetration may be precisely engineered by the diameter of the nozzle together with the energy and volume associated with the jet.

The main dose is then delivered through the track cut through the skin by the piston travelling down the syringe.

The pressure of the nozzle on the skin will produce a radial compressive stress pattern of approximately spherical symmetry. The compressive stress will collapse capillaries leading radially from the track cut through the skin. As the stress will decrease as approximately the inverse square of radius from the nozzle, the dose will be delivered to the end of the track.

1. An injector comprising a rigid tube (1) with an outlet at one end (46) and a non-return valve at the other end (46), a hole in the tube wall (3), an elastomeric liner (7) within the rigid tube (1) and a piston (26) arranged to impact the elastomeric liner (7) through the hole in the tube (3) to produce a high pressure transient.

2. An injector according to claim 1, including a spring member (20) arranged to act upon the piston (26) to cause the piston to impact the liner (7).

3. An injector according to claim 1 wherein the piston (26) is attached to a mass (24) which is capable of being accelerated.

4. An injector according to claim 3, wherein the mass (24) can be accelerated by a spring member (20).

5. An injector according to claims 4 wherein the spring (20) member is bi-stable.

6. An injector according to claim 5 wherein the spring member (20) can be manually energised to a latched position and triggered by pressure against the skin of a patient.

7. An injector according to any of claim 4, wherein the spring member (20) comprises an arcuate lamina which is deformable by bending to decrease in curvature.

8. An injector according claim 4 wherein one end of the spring member (21) is pivotable in a transverse channel (4) of the rigid tube (1).

9. An injector according claim 7 wherein the other end of the spring member (22) is connected to the mass (24).

10. An injector according claim 3 wherein the mass (24) is triangularly shaped.

11. An injector according to claim 10, wherein an apex (28) of the triangularly shaped mass is pivotable in a retaining groove (5) of the rigid tube (1).

12. An injector according to claim 3 wherein the mass (24) bears the piston (26) which fits within the hole (3) in the rigid tube for impacting the elastomeric liner (7).

13. An injector according to claim 1, wherein the elastomeric liner (7) is oversize or axially compressed to provide a seal within the rigid tube (1).

14. An injector according to claim 13 wherein the elastomeric liner is axially compressed between a shoulder (2) within the rigid tube (1) and an oversize plug (8) which comprises a channel (11) and is retained by friction therewithin.

15. An injector according to claim 14 wherein the plug (8) is a cylinder comprising one or more flats (9) or a helical groove, such that the fit of the plug within the bore (10) of the tube defines one or more capillary feed channels (11).

16. An injector according to claim 15 wherein the walls of the compressed elastomeric liner (7) cover the capillary channels (11) formed by the flats (9) or helical groove, to form a non-return valve biased in the closed position.

17. An injector according to claim 1 including an oversize capillary tube (44) which retains or compresses the elastomeric liner (7) and is itself retained by frictional forces.

18. An injector according to claim 17, including a blind elastomeric tube (7) with a transversely slot off-axis (42,43) which forms a non-return valve (46) with the retaining capillary tube (44).

19. An injector according to claim 18 wherein a conical cross-section (47) of the blind termination (41) of the elastomeric tube (7) biases the valve into a closed position.

20. An injector according to claim 17, wherein the capillary tube (44) is sharpened to pierce the rubber septum of an ampoule, to provide a supply of liquid drug to the tube.

21. An injector according to claim 1, wherein the elastomeric liner (7) is made of silicone rubber.

22. An injector according to claim 21 wherein the silicone rubber is filled with a proportion of silicone oil to provide intrinsic lubrication.

23. An injector according claim 1, wherein the outlet (15) is a nozzle.

24. An injector according claim 1, wherein the surface surrounding the outlet is saddle-shaped (50).

25. An injector according to claim 24, wherein the cross-section of the saddle-shaped outlet (50) is rectangular (56).

26. An injector according to claim 25, wherein the cross-section of the saddle-shaped outlet (56) increases in size with distance from the axis thereof (57).

27. An injector according claim 1, including a retractor spring (29) which partially removes the piston (26) from the hole (3) in the rigid tube wall (1), when the injector is in its quiescent state, so that the elastomeric liner (7) does not take a compression set.

28. An injector according to claim 27, wherein the retractor spring (29) is low rate and does not retract the piston beyond a given point, such that the elastomeric liner is not extruded through the hole (3) in the rigid tube wall by pressure from an associated injection syringe, in use.
